(19) United States
(12) Patent Application Publication
(10) Pub. No.: US 2007/0299380 A1
(43) Pub. Date: Dec. 27, 2007

(54) POSTURE AIDING METHOD AND APPARATUS

(76) Inventors: Serges J. Salivonchik, Nazareth, PA (US); Timothy J. Kern, Whitehall, PA (US)

Correspondence Address: DESIGN IP, P.C., 5100 W. TILGHMAN STREET, SUITE 205, ALLENTOWN, PA, 18104, US

(21) Appl. No.: 11/426,337

(22) Filed: Jun. 26, 2006

(51) Int. Cl. A61F 5/00

(52) U.S. Cl. 602/19; 602/5

(57) ABSTRACT

Posture correcting method and device fixed to the anatomy of a human user. The device is fixed at locations in the area of the upper cervical vertebrae and right and left scapular areas of the user.

### BACKGROUND OF THE INVENTION

Good posture is no longer considered an ideal but a necessity for proper health. The effects of bad posture go beyond the resultant muscle soreness and a hunched appearance of someone who is not standing or sitting upright. Poor posture has been identified as a source of reduced vital lung capacity, decreased GI function (including loss of bowel peristalsis), improper spinal function, disc damage, osteophyte formation, neurovascular entrapment, permanent changes in the musculoskeletal system, low energy and myofascial pain. In addition, poor posture correlates with the appearance of low self-esteem and the appearance of looking prematurely old. There is an inherent tendency for improper posture given that, as human beings, we tend to lean forward as our eyes focus our attention straight. We obviously reach and work in the forward position, often looking down. Changes in society and the workplace have resulted in sedentary lifestyles and occupations which cause individuals to engage in awkward repetitive motions, often sitting for excessive periods of time. As human beings, this is unnatural and leads to a multitude of problems. After hours of sitting, the shoulders round, the head comes forward and the lower back collapses. The combination of unnatural demands on the human body and poor mechanics has significant consequences. Poor posture and improper ergonomics at the work place are a major factor in myofascial pain. It is thought that myofascial pain accounts for 21% of visits to orthopedic clinics. It is estimated that 14% of the population suffers from chronic musculoskeletal pain and up to 60% of those have myofascial pain.

Proper muscular mechanics and well being depend in ideal posture. Ideal head, neck and shoulder posture occur when the ears align over the shoulders and the shoulders are back and not rolled forward. The cervical vertebrae display an inward “c-curve” in health. With poor posture, the curve is lost and a forward head position results. For every inch of forward head position, there is an additional 10 lbs of force on the neck, shoulders and spinal tissue. Unfortunately, the importance of good posture is undervalued and, since bad posture is gradual and habitual, the infrequent reminders to sit up straight are grossly inadequate.

An analysis of the prior art reveals two categories of posture-correcting devices. The first generally consists of braces, straps or bands that function by restricting movement away from improper posture. These can be complicated or difficult to apply and cumbersome to wear. The other type involves an inelastic woven a synthetic fabric whose length exceeds its width with adhesive on both ends. This can be attached to the body in two points while the individual is in a position of ideal posture. As the individual deviates from ideal posture, he or she is reminded by some discomfort in the form of pulling on the skin. This device is linear, affecting mainly one motion on one area of the body.

U.S. Pat. Nos. 5,107,826 4,995,383, 3,116,735 6,086,551 5,857,990 and pre-grant publications 2005/197607 and 2005/0192524 are representative of the prior art.

### BRIEF SUMMARY OF THE INVENTION

The present invention provides a method and apparatus for a user to maintain proper posture by aligning the ears over his or her shoulders.

After the device, according to one embodiment of the invention, is placed on a user's body, the user maintains a proper posture. As the user's neck deviates from a position of proper posture, the user will feel a slight tension in the skin area where the device is applied so that a proper posture can be resumed.

Therefore, in one aspect, the present invention is a posture aiding device comprising in combination; a generally “T” shaped thin, high strength, light weight, inelastic substrate, and a human compatible adhesive fixed to the outer portions of one surface of the “T” shaped substrate.

In another aspect, the present invention is a method of correcting the posture of an individual comprising the steps of; positioning the individual in a position where the individual's ears align over his or her shoulders, and applying a generally rigid posture aid to the individual's body, the device being attached at three separate locations in the individual's body, one location overlaying an upper cervical vertebrae and another location overlaying an upper left scapular area and another location overlaying the right scapular area of the individual's body.

In yet another aspect, the present invention is a posture aiding device comprising in combination; a thin, high strength, light weight, inelastic substrate with three extreme contact portions covering a generally triangular-shaped area, and a human compatible adhesive fixed to the extreme contact portions on one surface of said substrate.

### BRIEF DESCRIPTION OF THE DRAWINGS

FIG. 1 is a top plan view of a device according to the present invention.

FIG. 2 is a bottom plan view of the device of FIG. 1.

FIG. 3 is a schematic diagram of the device of FIG. 1 applied to the body of a user.

FIG. 4 is a left side view of the schematic on FIG. 3.

### DETAILED DESCRIPTION OF THE INVENTION

The present invention provides awareness to the user of ideal head, neck and shoulder posture by restricting the deviation from ideal posture of the neck and both shoulders. It does this by attaching at three points in a triangular fashion. The individual or an assistant adheres the upper adhesive end to the skin overlying the upper cervical vertebrae and the lower adhesive end, which consists of left and right sides, to the skin overlying the upper left and right scapular area. This is done while the head, neck and shoulders are in a position of ideal posture. This allows some freedom of motion; but, when the individual exceeds the parameters of ideal posture, they are reminded by a slight tension in the skin area intended to produce mild discomfort.

The invention is constructed as a simple, one-piece, easy-to-use, disposable device. It is made using a high strength, very thin, inelastic, non-woven polyester which is light weight and chameleon-like in color. The device has the shape of an upside-down letter “T.” The overall width exceeds the length. The wider, lower horizontal section (the top of the letter “T” before inversion), can have an hourglass shape with portions covered by a strong, medical grade, hypoallergenic adhesive on one side. The vertical portion of the “T” shape has adhesive on a wider end portion.

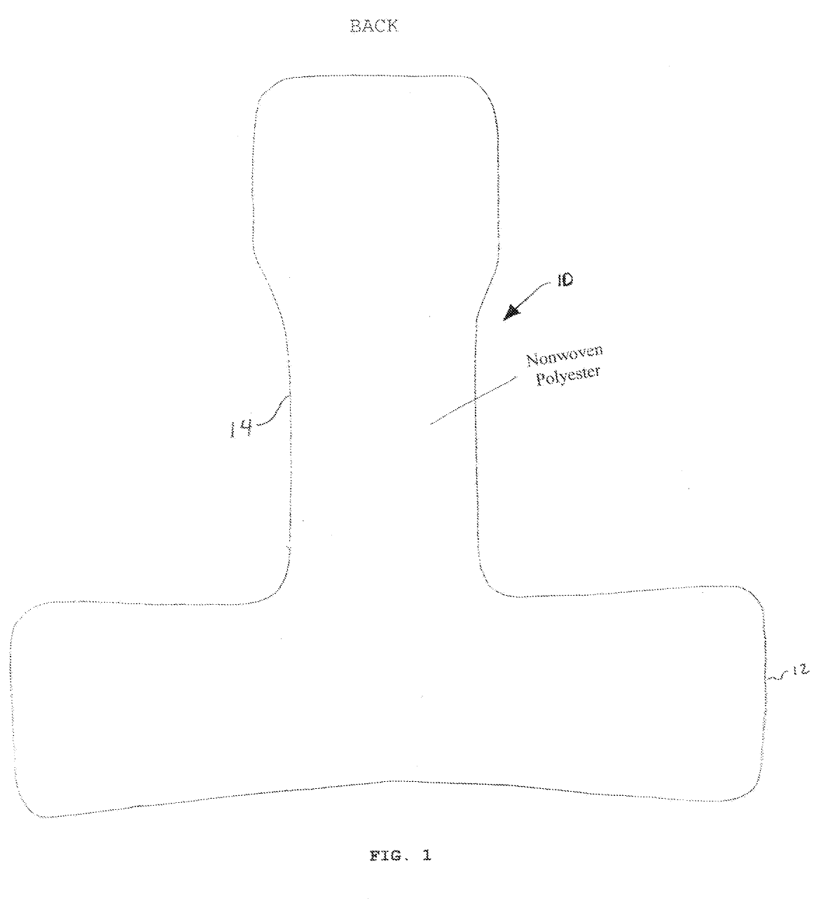

Referring to FIG. 1, the device 10 of the present invention is made of a thin, light weight, high strength, inelastic material substrate having the general shape of a “T.” As shown in FIG. 1, the “T” is inverted with the horizontal bar 12 on the bottom and the vertical bar 14 extending upwardly.

Device 10 can be fabricated from a non-woven polyester manufactured and sold by Posture Focus under the trade designation posture correcting pain reliever. The non-woven polyester has a minimum thickness of 0.022 inches.

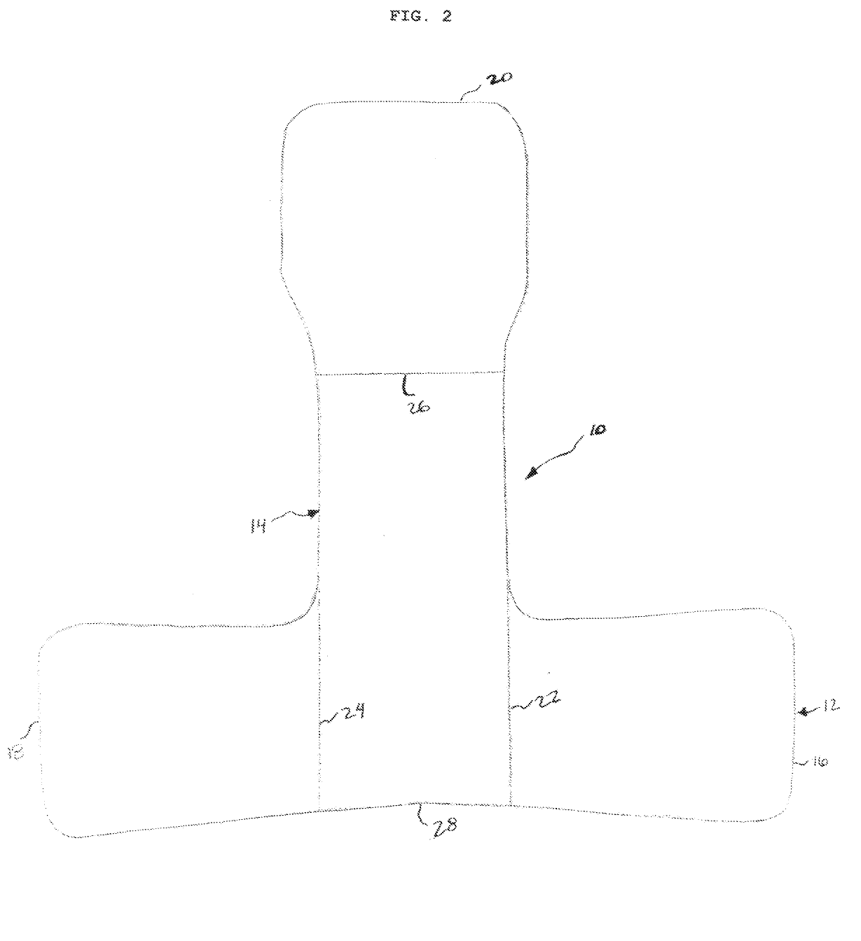

Referring to FIG. 2, the device 10 is presented on the reverse side with areas of adhesive covering the surface of the side of the device 10 extending from outward edges 16, 18, 20 to generally the locations 22, 24, 26 as shown on FIG. 2. If a substrate has a full-surface adhesive, the area marked by locations 22, 24 26, 28 can be covered with a non-adhesive layer.

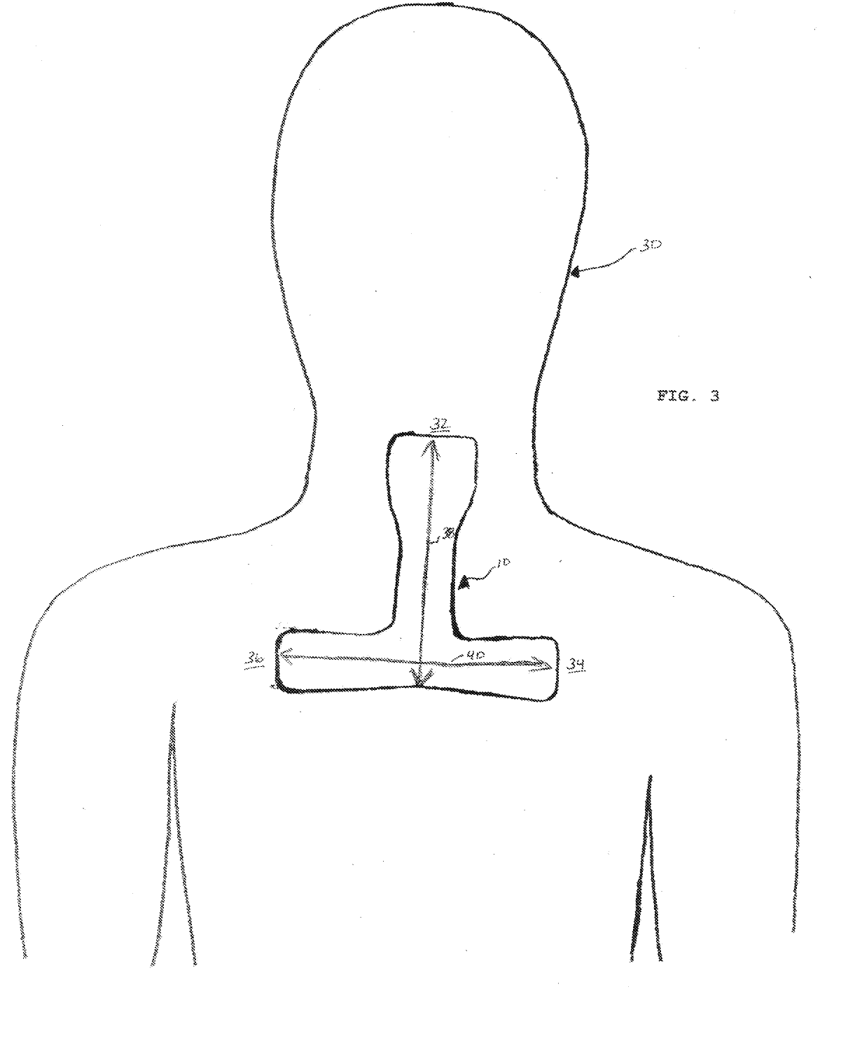

Referring to FIG. 3, the device 10 of FIG. 1 is placed on the body of a user 30 when the user is in a position of correct posture, when user's the ears are aligned over the shoulders, so that the adhesive portion 20, 26 overlays the upper cervical vertebrae of the user 30 in the location 32. The adhesive portions 16, 22 and 18, 24 are positioned to overlay the upper right scapular area 34 and upper left scapular area 36 of the user 30. Arrows 38-40 on device 10 illustrate the direction of the tension that will be applied to the device as the user deviates from a position of good posture when the user's head moves forward, left, right or in a circular movement.

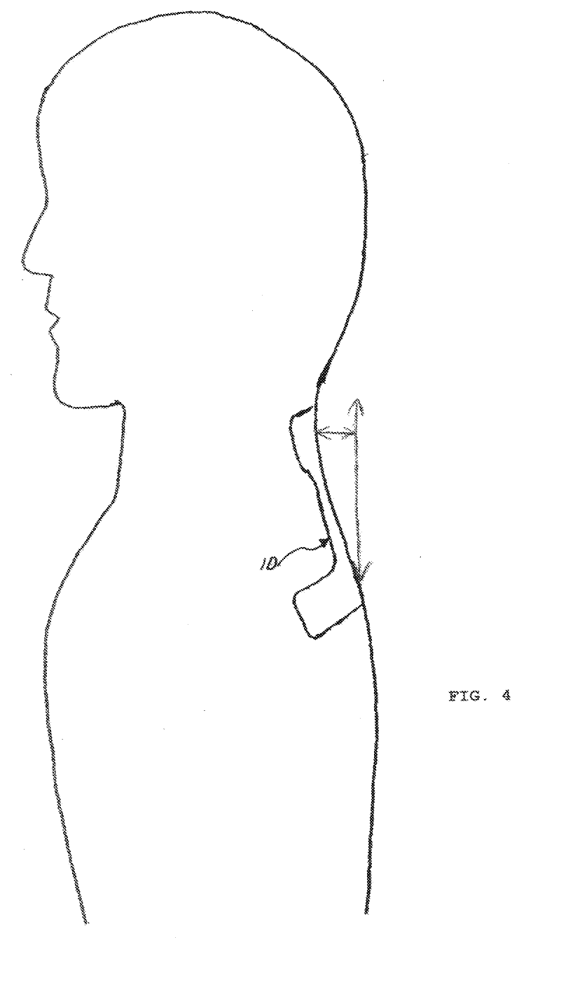

FIG. 4 illustrates potential force vectors 30, 40 as a function of the head movement of a user.

A device according the to invention was fabricated from non-woven polyester material having a horizontal bar 7 inches long from locations 16-18, a vertical bar 6.5 inches from locations 20 to 28 and a width of 2.25 inches. The adhesive pads were 2.5 inches long covering the surface of the substrate at the locations described above. The size of the device can be adjusted to fit any body type. The main feature is the three point fixing of the device at the locations discussed above. The device according to the invention could assume other shapes, e.g. a triangle.

There are two significant differences between the device of the present invention and the prior art. Most importantly, the device of the present invention attaches to three separate body parts, while the prior art device attaches to only two body parts along the midline (spine). Since two points make up a line, the result is only a linear vector of force functioning in 1 dimension. Since the device of the present invention is attached to 1 central location (skin over cervical spine) and two peripheral locations (skin over both scapula), the result is a plane of ideal posture which has vectors of force in two dimensions (as shown in FIGS. 4 and 5) Therefore, each point of contact has at least two vectors of force. For example, the shoulders are adducted (retracted posteriorly) at the same time they are elevated, preventing the downward, forward, slumped shoulder position; or, in the case of the neck, the head is pulled down and back as opposed to just down in the case of the more linear prior art example. Furthermore, given the effects of muscle tendons and ligaments the present invention approaches a 3 dimensional postural maintenance device. Second, ideal posture was previously described as the position where the ears align over the shoulders. Unlike the prior art, this device relates both shoulders to ideal head and neck position. This could not be accomplished with a linear device. The difference is like the difference of staking a tree with two instead of three stakes.

Having thus described our invention, what is desired to be secured by Letters Patent of the United States is set forth in the appended claims.

1. A posture aiding device comprising in combination:
a generally “T” shaped thin, high strength, light weight inelastic substrate; and
a human compatible adhesive fixed to the outer portions of one surface of the “T” shaped substrate.

2. A posture aiding device according to claim 1 wherein said substrate is fabricated from a non-woven polyester.

3. A posture aiding device according to claim 1 wherein said substrate is sized to that when applied to a user's body as an inverted “T” each end of a horizontal portion of said substrate is fixed to overlay opposite left and right scapular areas of said user and is positioned overlaying an upper cervical vertebrae of said user.

4. A posture aiding device according to claim 1 wherein said adhesive is medical grade, hypoallergenic adhesive.

5. A posture siding device according to claim 1 wherein said substrate is so constructed and arranged to have a width slightly greater than the length.

6. A method of correcting the posture of an individual comprising the steps of:
positioning the individual in a position of correct posture when the individual's ears align over his or her shoulders; and
applying a generally rigid posture aid to the individual's body, said device being attached at three separate locations on the individual's body, one location overlaying an upper cervical vertebrae and another location overlaying an upper left scapular area and another location overlaying an upper right scapular area of said individual's body.

7. A method according to claim 6 including the step of fabricating said posture correcting device a “T” shaped thin, high strength, light weight inelastic substrate; and a human compatible adhesive fixed to the outer portion of one surface of each of an end portion of each counter of the “T” shaped substrate.

8. A method according to claim 7 including the step of fabricating said posture correcting device from a high strength, light weight, thin, inelastic, non-woven polyester.

9. A posture aiding device comprising in combination;
a thin, high strength, light weight, inelastic substrate with three extreme contact portions covering a generally triangular-shaped area; and
a human compatible adhesive fixed to the extreme contact portions on one surface of said substrate.

10. A posture aiding device according to claim 1, wherein said substrate is fabricated from a non-woven polyester.
